(19) United States
(12) Patent Application Publication
(10) Pub. No.: US 2017/0049404 A1
(43) Pub. Date: Feb. 23, 2017

(54) Wearable LED Sensor Device Configured to Identify a Wearer's Pulse

(71) Applicant: Amiigo, Inc., Palo Alto, CA (US)

(72) Inventors: Ehsan Azarnasab, Palo Alto, CA (US); Abraham Carter, Palo Alto, CA (US); David Scott, North Salt Lake City, UT (US); Sameer Sontakey, Palo Alto, CA (US)

(21) Appl. No.: 15/221,445

(22) Filed: Jul. 27, 2016

Related U.S. Application Data
(60) Provisional application No. 62/207,268, filed on Aug. 19, 2015.

(51) Int. Cl. A61B 5/00; A61B 5/1455; A61B 5/08; A61B 5/024

(52) U.S. Cl. CPC A61B 5/7278; A61B 5/681; A61B 5/02433; A61B 5/14551; A61B 5/08; A61B 5/02405; A61B 5/7282

(57) ABSTRACT

A wearable sensor device can include at least one LED and a light sensor for generating a photoplethysmogram (“PPG”) from light emitted by the at least one LED. The wearable sensor device can also include a processing unit that is configured to process the PPG to produce a highly reliable representation of the wearer's pulse.

### CROSS-REFERENCE TO RELATED APPLICATIONS

The application claims priority to U.S. Provisional Patent Application No. 62/207,268 titled “Wearable Sensor Device Configured to Identify a Wearer's Pulse” which was filed Aug. 19, 2015.

### BACKGROUND

A wearable sensor device is a device worn by a user that is configured to monitor an action or characteristic of the user. For example, a wearable sensor device may include an accelerometer for detecting a user's movement and/or a biometric sensor for measuring the user's pulse rate. Many wearable sensor devices have been created that can track a wearer's pulse. However, such devices are typically limited to detecting the pulse rate and provide very little additional useful information. Although some devices have been produced for generating further details beyond pulse rate, the nature of wearable sensor devices make it difficult to generate reliable information.

### BRIEF SUMMARY

The present invention extends to wearable sensor devices that are configured to process a photoplethysmogram (PPG) and other pulse and heartbeat information to produce a highly reliable representation of the wearer's pulse. This processed PPG data (or “beat data”) can then be further analyzed to detect many different characteristics of the wearer's pulse which may represent that the wearer has a particular condition (e.g., an arrhythmia) or that the wearer is in a particular state (e.g., REM sleep).

In one embodiment, the present invention is implemented as a wearable sensor device that includes a housing configured to allow the wearable sensor device to be worn on a portion of the body, and a circuit that includes a first LED secured to the housing in a manner that causes the first LED to face the portion of the body when the wearable sensor device is worn; a light sensor secured to the housing, the light sensor being positioned to receive light that is transmitted from the first LED and reflected from or transmitted through the portion of the body, the light sensor being configured to generate a PPG representing the amount of light that is received by the light sensor over time; a processing unit configured to receive the PPG and to process the PPG, the processing of the PPG including: identifying peaks in the PPG; identifying valleys in the PPG; using the valleys to generate a base of the PPG; and subtracting the base from the PPG to yield beat data; and a storage for storing the beat data.

In another embodiment, the present invention is implemented as a method, performed by a wearable sensor device that includes at least one LED and at least one light sensor that generates a photoplethysmogram (“PPG”) from light emitted from one or more of the at least one LED, for generating beat data from the PPG. The PPG is received at a processing unit of the wearable sensor device. Peaks in the PPG are identified. Valleys in the PPG are also identified. The valleys are used to generate a base of the PPG. The base is subtracted from the PPG to yield the beat data. The beat data is then stored in a storage of the wearable sensor device.

In another embodiment, the acquired heartbeat information is used to derive the left ventricle ejection time (LVET), or to perform differential diagnosis of heart disease by distinguishing between conditions such as hypertrophy, cardio myopathy, aortic stenosis, hypertension, arrhythmia or low perfusion.

By using the derived measurements of cardiopulmonary health, sleep quality and surrogate measures of functional performance, physical capacity can be determined when compared to the other measurements taken at various physical activity levels.

This summary is provided to introduce a selection of concepts in a simplified form that are further described below in the Detailed Description.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an example of a bracelet that can be configured to implement embodiments of the present invention;

FIG. 2 illustrates an example circuit diagram for implementing embodiments of the present invention;

FIG. 3A illustrates an example of a PPG that can be generated by the light sensor of the circuit depicted in FIG. 2;

FIG. 3B illustrates an example of a hat that can be calculated for the PPG of FIG. 3A;

FIG. 3C illustrates an example of a base that can be calculated for the PPG of FIG. 3A;

FIG. 3D illustrates an example of beat data that is generated by subtracting the base from the PPG;

FIG. 3E illustrates an example of a smoothed and de-trended beat within the beat data of FIG. 3D; and

FIGS. 4A and 4B illustrate how beats per minute and pulse pressure values can be used to identify whether a peak in the base and hat represents an inhale or an exhale.

### DETAILED DESCRIPTION

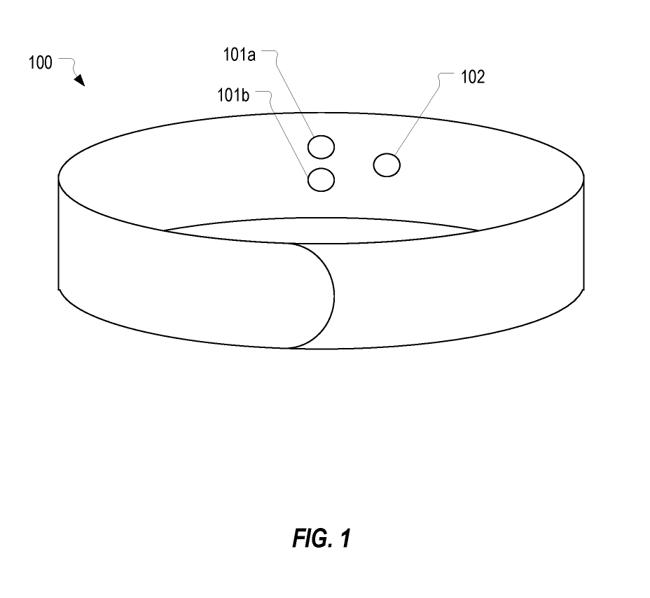

FIG. 1 illustrates an example of a bracelet 100 that can be configured to implement embodiments of the present invention. Although a bracelet configured to be worn around the wrist will be used to describe the present invention, it is noted that other types of wearable devices that can be worn on the wrist or other parts of the body can also be configured for use with or as embodiments of the present invention.

Bracelet 100 includes a red LED 101a and an infrared (IR) LED 101b that are exposed on an inner surface of bracelet 100. Accordingly, when bracelet 100 is worn by a wearer, red LED 101a and IR LED 101b will emit red light and infrared waves (collectively referred to as “light”) onto the wearer's skin. The use of two separate LEDs is only an example, and a wearable sensor device configured in accordance with embodiments of the present invention may equally include only a single LED or light source.

Bracelet 100 also includes a light sensor 102 that is exposed on the inner surface of bracelet 100. Light sensor 102 is positioned adjacent LEDs 101a, 101b so as to be able to capture light (i.e., both red light and infrared waves) that is emitted by LEDs 101a, 101b and reflected from the wearer's body. Alternatively, light sensor 102 could be positioned opposite LEDs 101a, 101b so as to detect light that is transmitted through the wearer's wrist. Accordingly, the present invention extends to wearable sensor devices that include one or more LEDs and one or more light sensors for sensing light that is either transmitted through or reflected by the wearer's skin. Light sensor 102 outputs a PPG representing the intensity of light that it receives over time. A PPG can be output for each of LEDs 101a, 101b.

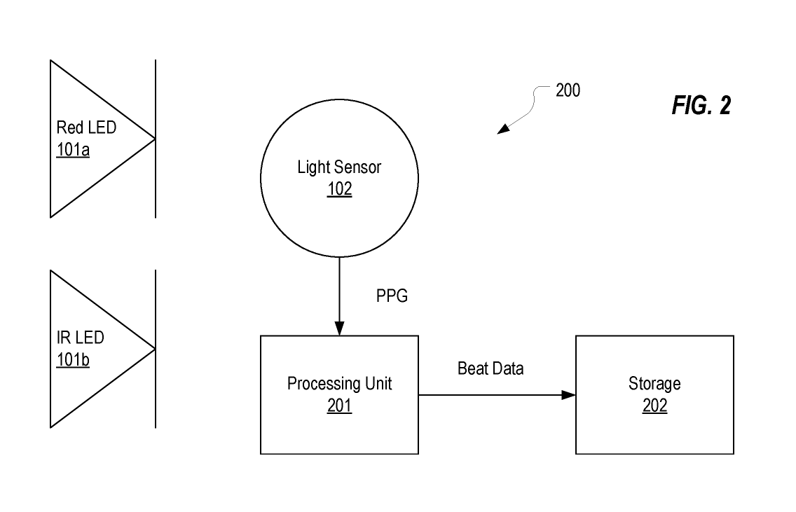

FIG. 2 illustrates a block diagram of a circuit 200 that can be employed within bracelet 100 in accordance with one or more embodiments of the present invention. Circuit 200 includes LEDs 101a, 101b, light sensor 102, processing unit 201, and storage 202. The PPG(s) that is/are output from light sensor 102 can be input to processing unit 201 to perform a number of processing steps which convert the PPG into a more useful form, i.e., into “beat data.” The beat data can then be stored for subsequent analysis as will be further described below.

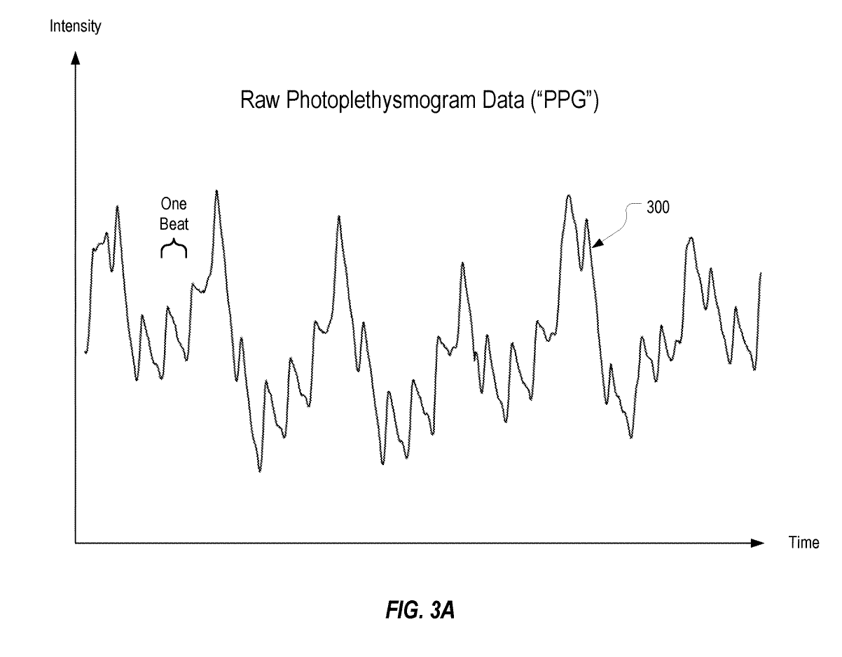

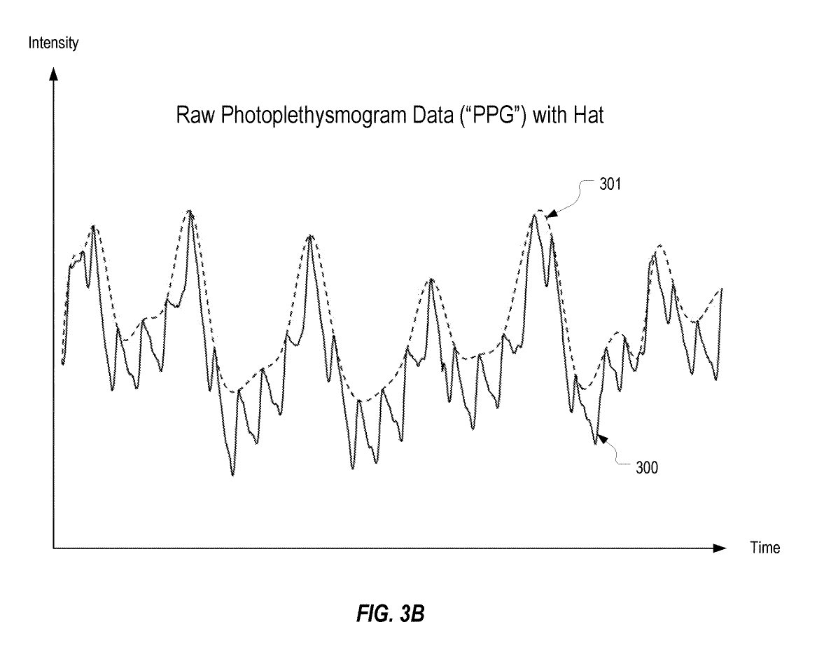

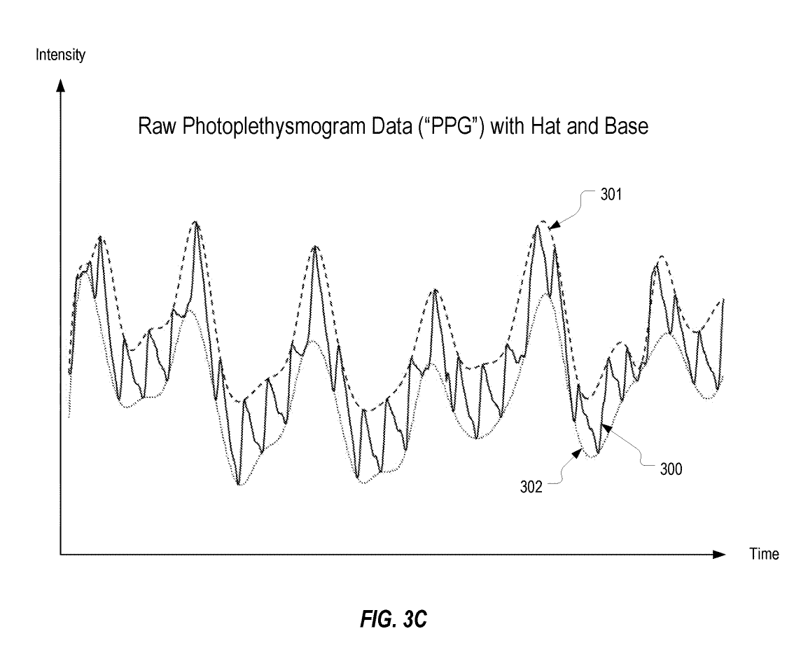

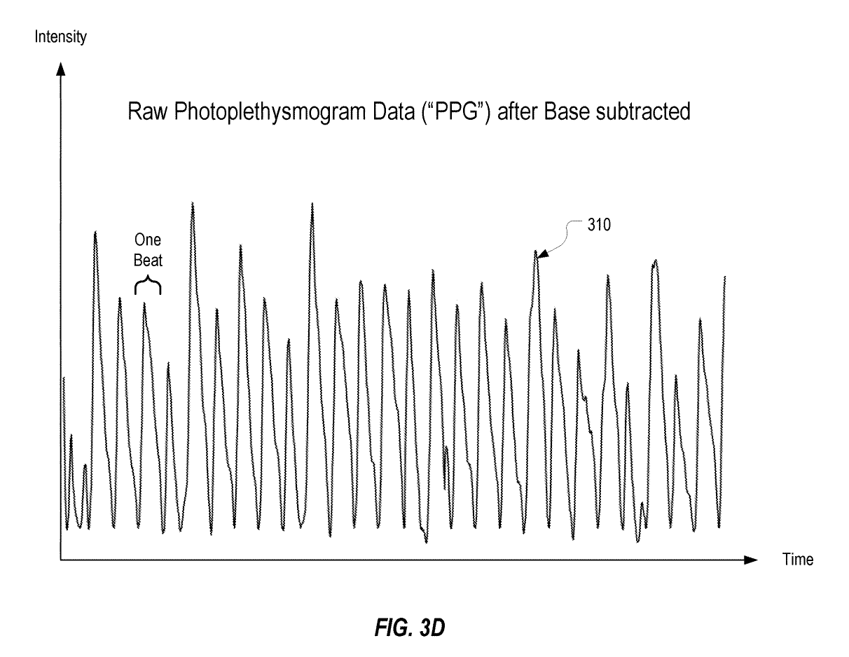

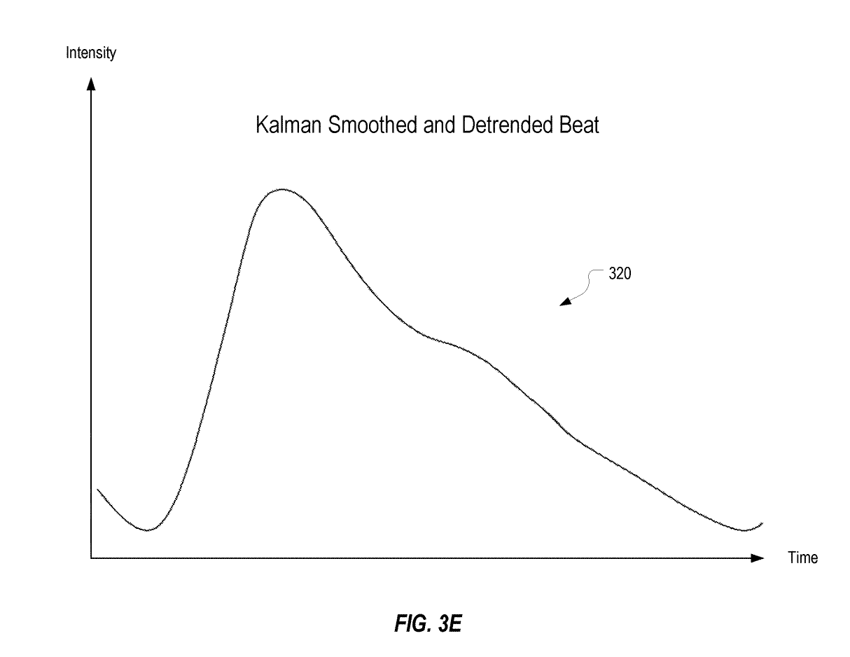

FIG. 3A illustrates a graph of an example PPG 300 that can be generated by light sensor 102 when bracelet 100 is worn. As shown, PPG 300 includes a number of pulses that each represents the occurrence of a heartbeat. However, these pulses include a significant amount of variability such as, for example, in their vertical positioning and overall shape. As was discussed in the background, this variability can be caused by a number of factors including, for example, the breathing pattern (which primarily causes the vertical movement of the PPG) or movement of the wearer. This variability in the PPG can make it difficult to extract useful information from the PPG.

In accordance with embodiments of the present invention, processing unit 201 can be configured to convert the PPG into beat data to facilitate the extraction of more useful information from the PPG. This conversion process may encompass a number of steps including: (1) identifying peaks in the PPG; (2) identifying valleys in the PPG; (3) validating the peaks and valleys; (4) generating a base from the valleys; (5) generating a hat from the peaks; and (6) generating the beat data.

To identify peaks in the PPG, wavelets may be employed. Wavelets are not sensitive to variations in the baseline of a signal, which variations are common in the PPG as shown in FIG. 3A. Wavelets are also capable of functioning on short-duration signals allowing beat data to be generated quickly when bracelet 100 is initially employed. After the peaks have been identified, the minimum value in the PPG between each peak can be identified as a valley. Accordingly, the result of this initial processing is an array of peak values and an array of valley values corresponding to a particular segment of the PPG.

In some embodiments, the peaks and valleys can be validated prior to commencing further processing. This validation can be performed using a model of the human pulse. For example, if the difference between two adjacent peaks or valleys exceeds what would be reasonable in view of the model of the human pulse, the corresponding portion of the PPG may be excluded from further processing. In this way, PPG data that is unreliable is prevented from influencing later analysis of the beat data.

The arrays of peak and valley values can then be used in a three-degree polynomial to generate a hat and base respectively for the PPG. FIG. 3B illustrates an example of a hat 301 (dashed line) that was generated for PPG 300. As shown, hat 301 generally extends from peak to peak in accordance with the three-degree polynomial. FIG. 3C illustrates an example of base 302 (dotted line) that was generated for PPG 300. Similar to hat 301, base 302 extends from valley to valley in accordance with the three-degree polynomial.

As stated above, the base generally represents the effects that breathing has on the PPG. More particularly, breathing directly alters blood volume which in turn alters the amount of light that is reflected by or transmitted through the blood. Therefore, the effects of breathing on the PPG must be removed in order to properly extract some heartbeat characteristics from the PPG. To accomplish this, the present invention subtracts the base from the PPG yielding a reliable representation of the wearer's pulse (or beat data 310) as shown in FIG. 3D.

In some embodiments, in addition to subtracting base 302 from PPG 300 to yield beat data 310, Kalman smoothing can be performed on beat data 310 and then each beat in beat data 310 can be linearly de-trended to produce a more accurate beat-shaped bellow such as is shown in FIG. 3E for one beat 320 of beat data 310.

Once beat data 310 has been generated, the values for each beat in beat data 310 (“individual beats”) can be stored (in storage 202). Each individual beat can then be evaluated to identify a number of beat model parameters for the individual beat including, for example, the foot of the beat, left ventricular ejection time onset, systolic ramp up, systolic peak, systolic ramp down, left ventricular ejection time offset, dicrotic notch, diastolic ramp up, diastolic peak, diastolic ramp down, etc. Although it is possible to estimate such parameters using PPG 300, the variability in PPG 300 makes such estimations difficult and inaccurate.

Accordingly, by generating beat data 310 as described above, the present invention greatly increases the accuracy of detecting such parameters. Any individual beat that appears to be invalid (i.e., any beat that does not fit within reasonable parameters of what a beat should look like) can be discarded to eliminate any potential that the invalid beat may degrade subsequent calculations of cardiovascular performance.

Once the beat data is generated, or more specifically, once the individual beats including their beat model parameters have been identified, the present invention can employ the beat data to identify different characteristics or states of the wearer. For example, the beat data can be evaluated to identify one or more patterns that are indicative of an arrhythmia Similarly, the beat data can be evaluated to identify when the wearer transitions between different stages of sleep. In another embodiment, the acquired heartbeat information is used to derive the left ventricle ejection time (LVET), or to perform differential diagnosis of heart disease by distinguishing between conditions such as hypertrophy, cardio myopathy, aortic stenosis, hypertension, arrhythmia or low perfusion.

By using the derived measurements of cardiopulmonary health, sleep quality and surrogate measures of functional performance, physical capacity can be determined when compared to the other measurements taken at various physical activity levels.

Although it is possible to perform such evaluations using the PPG directly, the evaluations are inaccurate and unreliable due to the high degree of variations that exist in the PPG that are not directly caused by the heart. Accordingly, the above described process of converting the PPG into beat data yields a highly reliable and accurate representation of the heart's performance This in turn enables a great number of evaluations to be easily and accurately performed using a portable and relatively simple wearable sensor device.

In addition to producing beat data 310 as described above, processing unit 210 can also be configured to employ hat 301 and base 302 to accurately detect other parameters such as oxygen saturation and breathing. As indicated above, in embodiments that include both red LED 101a and IR LED 101b, processing unit 210 can be configured to generate a hat and base for the PPG for each LED. These hats and bases will be referred to hereafter as hat base_{red}, hatᵢᵣ, and baseᵢᵣ. Processing unit 210 can also be configured to derive the oxygen saturation using these hats and bases in accordance with the following formula:

$$\frac{\frac{{hat}_{red} - {base}_{red}}{{base}_{red}}}{\frac{{hat}_{ir} - {base}_{ir}}{{base}_{ir}}}$$

To detect breathing, processing unit 210 can be configured to analyze the base and hat (e.g., using Bayesian frequency detection) to identify a recurring pattern. This recurring pattern is a repeating peak indicative of the occurrence of either an inhale or an exhale. From this processing, the wearer's respiration rate can be detected.

From the base and hat alone, it cannot be definitively determined when an inhale or exhale occurs since both will be represented as peaks in the hat and base. To address this, processing unit 210 can be configured to employ additional parameters so that peaks in the base and hat can be accurately identified as representing either an inhale or an exhale. For example, when an inhale occurs, a measurement of the beats per minute should be maximized while the pulse pressure value should be minimized Conversely, when an exhale occurs, the measurement of the beats per minute should be minimized while the pulse pressure value should be maximized

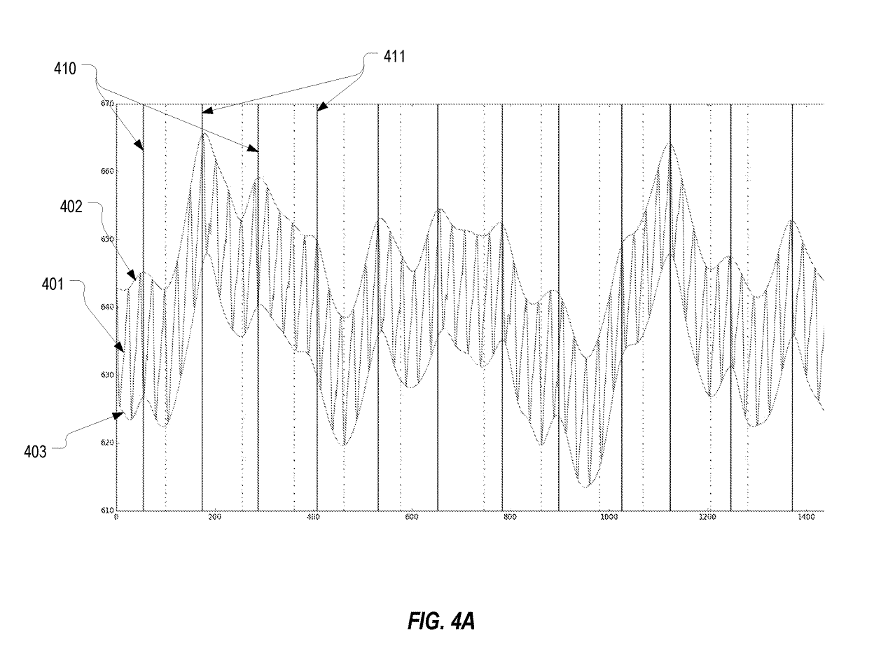

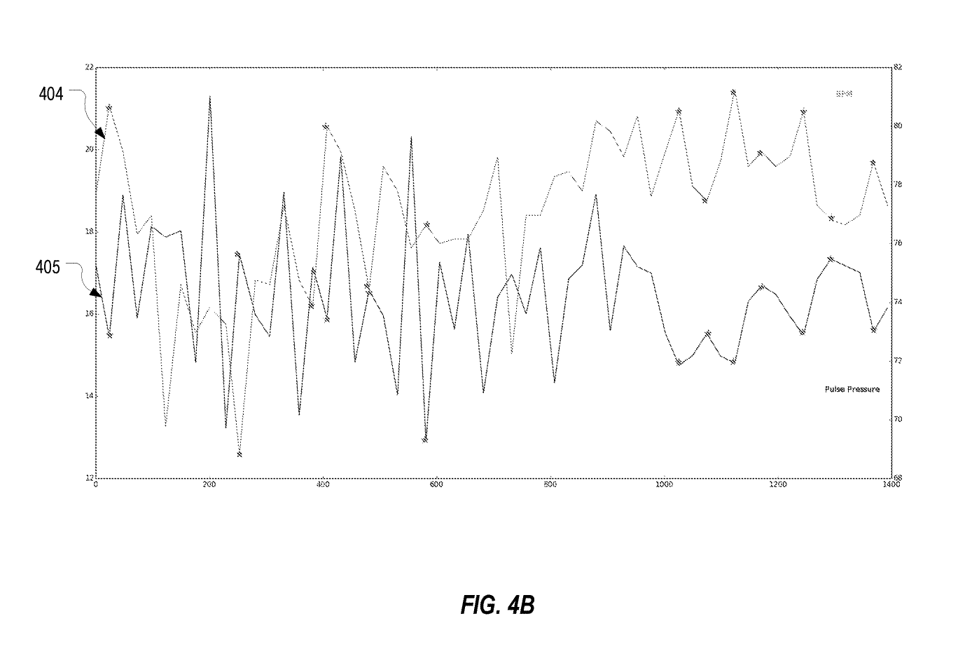

Accordingly, processing unit 210 can be configured to derive beats per minute and pulse pressure values from beat data 310 and use such values to identify whether peaks in the base and hat represent an inhale or an exhale. FIGS. 4A and 4B represent how this can be done. FIG. 4A is a graph of an example PPG 401 along with a corresponding hat 402 and base 403 which can be generated for PPG 401 as described above. The vertical black lines represent the peaks within the base and hat. FIG. 4B illustrates a graph a beats per minute 404 and pulse pressure 405 which were derived from PPG 401. The location of a peak in beats per minute 404 and the location of a valley in pulse pressure 405 (both of which are represented by stars in FIG. 4B) can be used to identify that a peak at the corresponding location in FIG. 4A represents the occurrence of an inhale. Similarly, a peak in pulse pressure 405 and a valley in beats per minute 404 (both of which are also represented by stars in FIG. 4B) can be used to identify that a peak at the corresponding location in FIG. 4A represents the occurrence of an exhale.

FIG. 4A includes black vertical lines indicating where peaks occur in hat 402 and base 403. Using beats per minute 404 and pulse pressure 405, these peaks can be identified as representing an inhale or exhale. For example, the peaks identified by lines 410 can be categorized as representing inhales, while the peaks identified by lines 411 can be categorized as representing exhales.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed:

1. A wearable sensor device comprising:
a housing configured to allow the wearable sensor device to be worn on a portion of the body; and
a circuit comprising:
a first LED secured to the housing in a manner that causes the first LED to face the portion of the body when the wearable sensor device is worn;
a light sensor secured to the housing, the light sensor being positioned to receive light that is transmitted from the first LED and reflected from or transmitted through the portion of the body, the light sensor being configured to generate a photoplethysmogram (“PPG”) representing the amount of light that is received by the light sensor over time;
a processing unit configured to receive the PPG and to process the PPG, the processing of the PPG including:
identifying peaks in the PPG;
identifying valleys in the PPG;
using the valleys to generate a base of the PPG; and
subtracting the base from the PPG to yield beat data; and
a storage for storing the beat data.

2. The wearable sensor device of claim 1, wherein the step of identifying peaks in the PPG comprises using wavelets to identify the peaks.

3. The wearable sensor device of claim 1, wherein the step of identifying valleys in the PPG comprises identifying a minimum value in the PPG between each adjacent set of peaks.

4. The wearable sensor device of claim 1, wherein the base is generated from the valleys using a three-degree polynomial.

5. The wearable sensor device of claim 1, wherein the processing of the PPG further includes:
using the peaks to generate a hat of the PPG.

6. The wearable sensor device of claim 5, wherein the hat is generated from the peaks using a three-degree polynomial.

7. The wearable sensor device of claim 5, wherein the processing of the PPG further includes:
subtracting the base from the hat then dividing by the base to yield a result representing oxygen saturation.

8. The wearable sensor device of claim 5, wherein the processing of the PPG further includes:
identifying a recurring pattern in the base and the hat, the recurring pattern representing the occurrence of a breath.

9. The wearable sensor device of claim 7, further comprising:
generating a beats per minute value or a pulse pressure value; and
validating one or more of peaks or valleys in the result using the beats per minute value or the pulse pressure value.

10. The wearable sensor device of claim 1, wherein the processing of the PPG further includes:
applying Kalman smoothing to the beat data; and
de-trending the beat data.

11. The wearable sensor device of claim 1, wherein the processing of the PPG further includes:
storing individual beats of the beat data.

12. The wearable sensor device of claim 11, wherein each individual beat includes values identifying a number of beat model parameters for the individual beat.

13. The wearable sensor device of claim 1, wherein the circuit further comprises:
a second LED secured to the housing in a manner that causes the second LED to face the portion of the body when the wearable sensor device is worn;
wherein the light sensor being positioned to receive light that is transmitted from the second LED and reflected from or transmitted through the portion of the body, the light sensor being configured to generate a second PPG representing the amount of light from the second LED that is received by the light sensor over time;
wherein the processing unit is configured to receive the second PPG and to process the second PPG, the processing of the second PPG including:
identifying peaks in the second PPG;
identifying valleys in the second PPG;
using the valleys in the second PPG to generate a base of the second PPG; and
subtracting the base of the second PPG from the second PPG to yield second beat data; and
wherein the storage stores the second beat data.

14. The wearable sensor device of claim 13, wherein the first LED is a red LED and the second LED is an infrared LED.

15. The wearable sensor device of claim 14, further comprising:
generating a value representing oxygen saturation using the base and hat of the PPG and the base and hat of the second PPG.

16. The wearable sensor device of claim 14, further comprising:
generating a value representing breathing using the base and hat of the PPG and the base and hat of the second PPG, and using the value to compute beats per minute and pulse pressure variations.

17. The wearable sensor device of claim 1, wherein the processing of the PPG further includes:
comparing the peaks or valleys to a known model of the human pulse; and
discarding a portion of the PPG corresponding to a peak or valley based on the comparison.

18. A method, performed by a wearable sensor device that includes an LED and a light sensor that generates a photoplethysmogram (“PPG”) from light emitted from the LED, for generating beat data from the PPG, the method comprising:
receiving the PPG at a processing unit of the wearable sensor device;
identifying peaks in the PPG;
identifying valleys in the PPG;
using the valleys to generate a base of the PPG;
subtracting the base from the PPG to yield the beat data; and
storing the beat data in a storage of the wearable sensor device.

19. The method of claim 18, wherein identifying peaks in the PPG comprises using wavelets to identify the peaks.

20. The method of claim 18, further comprising:
using the peaks to generate a hat of the PPG.

21. The method of claim 18, further comprising:
subtracting the base from the hat then dividing by the base to yield a result representing oxygen saturation.

22. The method of claim 18, further comprising:
identifying a recurring pattern in the base and the hat, the recurring pattern representing the occurrence of a breath.

23. The method of claim 18, wherein storing the beat data comprises:
storing individual beats of the beat data, each individual beat including values identifying a number of beat model parameters for the individual beat.

24. A method for acquiring heartbeat information to perform differential diagnosis of heart disease; comprising:
attaching a wearable sensing device to a user, the sensing device having an LED and a light sensor;
generating a photoplethysmogram (PPG) from light emitted from the LED; and
deriving beat data from the PPG to perform differential diagnosis of heart disease by distinguishing between conditions such as hypertrophy, cardiomyopathy, aortic stenosis, hypertension, arrhythmia and low perfusion and to derive left ventricle ejection time.
